SEQUENCE LISTING (4 sequences)

SEQ ID NO: 1 - DNA, 24 bp, Artificial
  Chemically synthesized
gcttttcagggactatgcaatgaa

SEQ ID NO: 2 - DNA, 24 bp, Artificial
  Chemically synthesized
aagggcagtgacactggtttgaga

SEQ ID NO: 3 - DNA, 20 bp, Artificial
  Chemically synthesized
agggaaacggcctttctgaa

SEQ ID NO: 4 - DNA, 20 bp, Artificial
  Chemically synthesized
gtggagaagacgacgatttc

(19) United States
(12) Patent Application Publication
(10) Pub. No.: US 2007/0065833 A1
(43) Pub. Date: Mar. 22, 2007

(54) Method of detecting thyroid cancer

(75) Inventor: Manjula Gupta, Beachwood, OH (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: The Cleveland Clinic Foundation

(21) Appl. No.: 11/201,508

(22) Filed: Aug. 11, 2005

Related U.S. Application Data
(60) Provisional application No. 60/600,589, filed on Aug. 11, 2004.

(51) Int. Cl. C12Q 1/68

(52) U.S. Cl. 435/6

(57) ABSTRACT

A method of detecting thyroid cancer in a subject includes obtaining a nucleic acid sample from a bodily sample of the subject and determining whether the nucleic acid sample contains at least one of thyroid stimulating hormone receptor (TSHR) mRNA or thyroglobulin (Tg) mRNA.

### RELATED APPLICATION

The present application claim priority from U.S. Provisional Patent Application Ser. No. 60/600,589, filed Aug. 11, 2004, herein incorporated by reference in its entirety.

### FIELD OF THE INVENTION

The present application relates to a method of detecting thyroid cancer in a subject and to method of detecting thyroid cancer using a nucleic acid based assay.

### BACKGROUND OF THE INVENTION

It is estimated that between 4-7% of the population harbors thyroid nodules. Literature reports that 5-30% of these nodules are malignant. The main diagnostic consideration in these cases is the exclusion of malignancy. Currently, the method offering the best preoperative prediction of the nature of these nodules is the fine needle aspiration biopsy of the lesion (FNA). Use of FNA resulted in decrease in number of thyroidectomies performed and increase in the yield of malignancy in resected lesions. However, instances of inadequate sampling of the lesion and overlapping cytological features of benign and malignant thyroid neoplasms are inherent limitations of this technique. The major FNA limitation is its inability to distinguish between well-differentiated follicular carcinomas and benign follicular adenomas. Patients with follicular thyroid neoplasm usually undergo thyroidectomy and about 15% have malignant lesion. Reliable preoperative diagnosis of benign lesion would greatly reduce number of unnecessary surgeries.

Cancer cells circulate in the peripheral blood and lymphatic channels prior to developing metastasis at distant site. Detection of these cells provides a tool for early diagnosis of cancer and its metastatic potential. RT-PCR is a sensitive and powerful technique in detecting the presence of specific cell type in circulation based on its ability to identify tissue/tumor specific mRNA transcripts. The reverse transcription polymerase chain reaction (RT-PCR) of specific thyroid marker, Tg, has been utilized to detect circulating thyroid cancer cells. Most investigators have detected circulating Tg mRNA in normal subjects thus limiting its use only to detect residual/recurrent thyroid cancer in thyroidectimized patients. The success of a PCR-based assay depends largely on a combination of the following factors: the sample processing procedure, the purity of RNA, the location of PCR primers, the cycling conditions, and the signal detection methods.

### SUMMARY OF THE INVENTION

An aspect of the present invention relates to a method of detecting thyroid cancer in a subject. The method comprises obtaining a nucleic acid sample from a bodily sample of the subject and determining whether the nucleic acid sample contains thyroid stimulating hormone receptor (TSHR) mRNA. The THSR mRNA can be determined by amplifying a segment of TSHR mRNA in the nucleic acid sample and detecting the presence of the amplified portion of the TSHR mRNA. The amplification can be performed with a pair of primers that are complementary to the TSHR mRNA transcripts. In an aspect of the invention, the primer pair can have nucleotide sequences comprising respectively SEQ ID NO: 1 and SEQ ID NO: 2.

Another aspect of the invention relates to a preoperative assay for determining whether thyroid neoplasia in a subject is benign or malignant. The preoperative assay comprises obtaining a nucleic acid sample from a bodily sample of the subject and determining whether the nucleic acid sample contains thyroid stimulating hormone receptor (TSHR) mRNA. The THSR mRNA can be determined by amplifying a segment of TSHR mRNA in the nucleic acid sample and detecting the presence amplified portion of the TSHR mRNA. The amplification can be performed with a pair of primers that are complementary to the TSHR mRNA transcripts. In an aspect of the invention, the primer pair can have nucleotide sequences comprising respectively SEQ ID NO: 1 and SEQ ID NO: 2.

A further aspect of the invention relates to a kit for detecting thyroid cancer in a subject. The kit comprise a pair of primers capable of amplifying a segment of TSHR mRNA. The amplified segment can include at least a portion of exons 6-9 of TSHR mRNA. In an aspect of the invention, the primers can comprise at least 10 contiguous nucleotides can have nucleotide sequences comprising respectively SEQ ID NO: 1 and SEQ ID NO: 2

Another aspect of the invention relates to a preoperative assay for determining whether thyroid neoplasia in a subject is benign or malignant. The preoperative assay comprises obtaining a nucleic acid sample from a bodily sample of the subject and determining whether the nucleic acid sample contains thyroglobulin mRNA. The Tg mRNA can be determined by amplifying a segment of Tg mRNA in the nucleic acid sample and detecting the presence amplified portion of the Tg mRNA. The amplification can be performed with a pair of primers that are complementary to the Tg mRNA transcripts. In an aspect of the invention, the primer pair can have nucleotide sequences comprising respectively SEQ ID NO: 3 and SEQ ID NO: 4.

A further aspect of the invention relates to a kit for detecting thyroid cancer in a subject. The kit comprise a pair of primers capable of amplifying a segment of Tg mRNA. The amplified segment can include at least a portion of exons 1-5 of Tg mRNA. In an aspect of the invention, the primers can comprise at least 10 contiguous nucleotides can have nucleotide sequences comprising respectively SEQ ID NO: 3 and SEQ ID NO: 4

### BRIEF DESCRIPTION OF THE DRAWINGS

The foregoing and other aspects of the present invention will become apparent to those skilled in the art to which the present invention relates upon reading the following description with reference to the accompanying drawings.

FIG. 1 illustrates a representative gel picture showing RT-PCR results for thyroglobulin (Tg), TSHR and for glyceraldehydes 3-phosphate dehydrogenase (GAPDH) in nine patient samples (Lanes 1-9), one negative control (no reverse transcription; Lane 10) and one positive control (thyroid cancer tissue RNA: Lane 11). Lanes 1, 5 and 6 benign thyroid disease patients; Lanes 2 and 4 thyroid cancer patients with no evidence of disease; Lanes 3,7-9 thyroid cancer patients with evidence of disease.

FIG. 2 illustrates a representative gel picture showing RT-PCR results for Tg and TSHR in patients (lanes 2-8). Positive control is in lane 1, and negative control is in lane 9.

FIG. 3 illustrates RT-PCR results in 18 patients with nondiagnostic FNA cytology *, Two thyroiditis and one colloid nodule. FP, False positive (hyperplastic oxyphilic nodule); HA, Hurthle cell adenoma

### DETAILED DESCRIPTION

The present invention may be understood more readily by reference to the following detailed description of the embodiments of the invention, and to the Examples and sequence listings included herein.

As used herein in the specification and the claims, the following terms have the given meaning unless expressly stated to the contrary.

The phrase “specific to”, “specific for”, and “unique to” the TSHR mRNA or Tg mRNA as used herein in relation to a nucleic acid or nucleic acid fragment means a nucleic acid or nucleic acid fragment that is not common to other mRNA.

The term “fragment” as used herein in relation to a nucleic acid means a sub-sequence of a nucleic acid that is of a sufficient size and confirmation to properly function as, for example, a hybridization primer in a polymerase chain reaction (PCR) or in another manner characteristic of nucleic acids.

The term “isolated” means that the nucleic acids or nucleic acid fragments are of sufficient purity so that they may be employed, and will function properly, in a clinical diagnostic, experimental or other procedure, such as a hybridization assay or an amplification reaction for TSHR mRNA or Tg mRNA. Many procedures are known by those of ordinary skill in the art for purifying nucleic acids, nucleic acid fragments, and materials with which they may normally be associated prior to their use in various procedures.

The term “substantially similar” in relation to the nucleic acid sequences of the present invention, or to the nucleotide sequences complementary the nucleotide sequences of the present invention, refers to a nucleic acid which is similar to the to the nucleic acid sequences of the present invention, or to nucleic acid sequences complementary to the nucleic acid sequences of the present invention, and which retains the functions of such nucleic acid, but which differs from such nucleic acid by the substitution, deletion, and/or addition of one or more nucleotides, and/or by the incorporation of some other advantageous feature. Nucleotide sequences of the present invention are substantially similar to a nucleic acid sequence if these percentages are from 100% to 80% or from 0 base mismatches in a 10 nucleotide sequence to 2 bases mismatched in a 10 nucleotide sequence. In some embodiments, the percentage is from 100% to 85%. In other embodiments, this percentage is from 90% to 100%; in still other embodiments, this percentage is from 95% to 100%.

The present invention provides a method of detecting thyroid cancer in a subject. The method can be used as a preoperative assay (e.g., prior to a thyroidectomy) for determining whether thyroid neoplasia in a subject is benign or malignant. The method can also be used as a post operative assay for monitoring metastatic thyroid cancer recurrence following thyroidectomy.

In accordance with an aspect of the invention, the method can comprise detecting circulating thyroid cells in a bodily sample of a subject by obtaining an appropriate nucleic acid sample from the bodily sample of the subject and determining whether the nucleic acid sample contains thyroid stimulating hormone receptor (TSHR) mRNA.

As described herein, “bodily sample” includes any bodily sample (e.g., fluid) from the body of the subject that one can obtain a nucleic acid sample so as to determine whether the nucleic acid sample contains the marker sequence. One such example is blood, specifically peripheral blood. However, one skilled in the art could obtain other bodily samples, such as fine needle aspirates and bronchial fluids, that would be able to determine whether the sample contains TSHR mRNA so as to determine whether circulating thyrocytes exist.

One skilled in the art could be able to practice the subject invention in various subjects, e.g., animals, but specifically humans.

The presence TSHR mRNA can be determined by detecting a target nucleotide sequence of the TSHR mRNA. It has been found that at least a portion of the nucleic acid sequence that comprises mRNA corresponding to the reverse transcript of DNA encoding TSHR can be used as a target nucleotide sequence for nucleic acids utilized in detection and differentiation of thyroid neoplasia. The phrase “target nucleotide sequence” refers to a region of a nucleotide, which is to be amplified, detected, or otherwise analyzed. By way of example, a portion of the nucleic acid sequence that comprises TSHR mRNA, which can be used as a target nucleotide sequence in accordance with the present invention, comprises exons 6 to 9 of TSHR mRNA.

In an aspect of the invention, the target nucleotide sequence can be detected by amplifying a target nucleotide sequence of the nucleic acid sequence that comprises mRNA corresponding to the reverse transcript of DNA encoding TSHR and detecting the amplified target sequence. The target nucleotide sequence of the present invention can be amplified by selectively hybridizing oligonucleotide primers that facilitate transcription and replication of at least a portion of the TSHR mRNA (or a reverse transcript of the TSHR mRNA). The term “hybridize” as used herein refers to the formation of a duplex structure by two single-stranded nucleic acids due to fully (100%) or less than fully (less than 100%) complementary base pairing. Hybridization can occur between fully and complementary nucleic acid strands, or between less than fully complementary nucleic acid strands which contain regions of mismatch due to one or more nucleotide substitutions, deletions, or additions.

The oligonucleotide primers of the present invention serve as a priming position or initiation position for the action of primer dependent polymerase activity. The oligonucleotide primers include nucleic acid sequences that are specific TSHR mRNA and that can be used to amplify a target nucleotide sequence. The target nucleotide sequence is defined by contiguous nucleotides of TSHR mRNA, such as the nucleotides of the exons 6-9 of TSHR mRNA.

The oligonucleotide primers of the present invention can comprise a pair of oligonucleotide primers that hybridize to nucleotide sequences, which flank the target nucleotide sequence, so that synthesis by the action of a polymerase, such as Taq polymerase, proceeds through the region between the two primers. This is advantageous because after several rounds of hybridization and replication the amplified target nucleotide sequence produced is a segment having a defined length whose ends are defined by the sites to which the primers hybridize.

Oligonucleotide primers capable of specifically (or selectively) hybridizing to the TSHR mRNA in accordance with the present invention can comprises at least about 10 nucleotides. By way of example, the oligonucleotide primers can comprise about 10 to about 40 nucleotides, and more particularly about 15 to about 35 nucleotides. The oligonucleotide primers can be of sufficient length and complementary with a portion of the nucleotide sequence of the TSHR mRNA to form a duplex with sufficient stability for the purpose intended. For example, the oligonucleotide primers should contain a nucleic acid sequence of sufficient length and complementarity to the targeted TSHR mRNA to allow the polymerizing agent to continue replication from the primers, which are in stable duplex form with the target sequence, under polymerizing conditions.

An example of a pair of nucleic acid sequences that can be used for the pair of nucleotide primers include SEQ ID NOs: 1 and 2. SEQ ID NO: 1 is a forward primer that comprises the following nucleotide sequence: 5′GCTTTTCAGGGACTATGCAA-TGAA 3′. SEQ ID NO: 2 is a reverse primer that comprises the following nucleotide sequence: 3′AGAGTTTGGTCAC-AGTGACGGGAA 5′. SEQ ID NOs: 1 and 2 when used as oligonucleotide primers are capable of amplifying a 212 base pair segment of exons 6-9 of TSHR mRNA.

It will be appreciated by one skilled in the art that other oligonucleotide primers of the present invention can include nucleic acid sequences complementary to SEQ ID NOs: 1-2, nucleic acid sequence substantially similar to SEQ ID NOs: 1-2, nucleic acid sequences substantially similar to a nucleic acid sequence complementary to SEQ ID NOs: 1-2, a fragment of SEQ ID NOs: 1-2 that specifically hybridize to the TSHR mRNA, a fragment of a nucleic acid sequence complementary to SEQ ID NOs: 1-2 that specifically hybridize to TSH mRNA, a fragment of a nucleic acid sequence substantially similar to SEQ ID NOs: 1-2 that specifically hybridizes to the TSHR mRNA, and a fragment of a nucleic acid sequence substantially similar to nucleic acid sequences complementary to SEQ ID NOs: 1-2 that specifically hybridizes to the TSHR mRNA. It will also be appreciated that the oligonucleotide primers can include other nucleic acid sequences as long as these nucleic acid sequences specifically hybridize to TSHR mRNA, and specifically amplify exons 6-9 of the TSHR mRNA.

The nucleic acids used to form the TSHR mRNA oligonucleotide primers in accordance with the present invention can be derived from TSHR mRNA. The derived nucleic acid is not necessarily physically derived from TSHR mRNA, but may be generated in any manner including, for example, chemical synthesis, DNA replication, reverse transcription, or transcription as well as generated from RNA and peptide nucleic acids (PNAs).

The TSHR mRNA oligonucleotide primers in accordance with the present invention may be made by methods well known in the art, such as chemical synthesis. The TSHR mRNA oligonucleotide primers may be synthesized manually or by machine. They may also be synthesized by recombinant methods using products incorporating viral and bacterial promoters.

The TSHR mRNA oligonucleotide primers can be used in an amplification assay that detects at least a portion of the TSHR mRNA. In an aspect of the invention the oligonucleotide primers can be used in a polymerase chain reaction (PCR) assay. In a PCR assay nucleic acids of a bodily sample is contacted with oligonucleotide primers that are specific to TSHR mRNA and that can be used to amplify a target nucleotide sequence. The target nucleotide sequence can be defined, for example, by contiguous nucleotides from exons 6-9 of TSHR mRNA. PCR amplification is then conducted on the resulting mixture using a temperature program and for a number of thermal cycles sufficient to amplify the target nucleotide sequence of TSHR mRNA, if present. The PCR amplification can be carried out in any commercially available PCR thermal cycling apparatus. For example, the PCR amplification can be performed using rapid temperature cycling techniques. Rapid temperature cycling techniques use a high surface area-to-volume sample container, such as a capillary tube, to contain the reaction amplification sample. The use of a high surface-area-to-volume sample container allows for rapid temperature response and temperature homogeneity throughout the sample. Rapid temperature cycling is contrasted to conventional temperature cycling in that 30 cycles of amplification can be completed in 15 minutes and the resulting PCR amplification products contain fewer side products. Thus, with rapid temperature cycling techniques the required times for amplification are reduced approximately ten-fold, and specificity is improved.

It will appreciated by one skilled the art that the THSR mRNA primers as well as other nucleic acids complementary to TSHR mRNA nucleic acids can also be used in other assays, such as a RAPD assay or other amplification assay.

The amplified target nucleotide sequence, if present, can then detected using known detection techniques. These detection techniques can be qualitative and/or quantitative. Examples of detection techniques that can be used in accordance with the present invention include visualization of restriction enzyme digestion patterns determined by gel electrophoresis, sequencing of the amplified target nucleotide sequence, detection of the amplified nucleotide sequence with an oligonucleotide hybridization probe. Copy number and quantitation can be performed by standard hybridization procedures such as Southern or Northern analysis. If an oligonucleotide hybridization probe is used for detection, the oligonucleotide hybridization probe can include a pair of nucleic acid sequences that are labeled with a fluorescence resonance energy transfer (FRET) pair.

When the detection method (e.g., melting point analysis) produces a result indicating that target nucleotide sequence amplified by the oligonucleotide primers is present, it is concluded that the original sample contains TSHR mRNA. Conversely, if no evidence of the target nucleotide sequence is detected, it is concluded that sample is free of TSHR.

Optionally, the polymerase chain reaction (PCR) amplification step and the detection step of the method are performed essentially simultaneously. The essentially simultaneous PCR amplification step and the detection step are performed in an apparatus that includes a rapid temperature cycler component and a fluorescent detection component. An example of such a device is described in U.S. Pat. No. 6,140,540, the disclosure of which is incorporated herein by reference. A preferred device that includes a rapid cycler component and fluorescent detection component is commercially available from Roche Molecular Biochemicals, of Indianapolis, Ind. under the trade name LIGHTCYCLER.

The level of TSHR mRNA detected in the bodily sample obtained from the test subject can be compared to a predetermined value. The predetermined value can be based upon the levels of TSHR mRNA in comparable samples obtained from the general population or from a select population of human subjects. For example, the select population may be comprised of apparently healthy subjects. “Apparently healthy”, as used herein, means individuals who have not previously had any signs or symptoms indicating the presence of disease, such as thyroid cancer. In other words, such individuals, if examined by a medical professional, would be characterized as healthy and free of symptoms of disease.

The predetermined value can be related to the value used to characterize the level of TSHR mRNA in the bodily sample obtained from the test subject. Thus, if the level of TSHR mRNA is an absolute value, the predetermined value is also based upon the units of TSHR mRNA in individuals in the general population or a select population of human subjects. Similarly, if the level of TSHR mRNA is a representative value such as an arbitrary unit, the predetermined value is also based on the representative value.

The predetermined value can take a variety of forms. The predetermined value can be a single cut-off value, such as a median or mean. The predetermined value can be established based upon comparative groups such as where the level of systemic marker (e.g., level of TSHR mRNA) in one defined group is double the level of systemic marker in another defined group. The predetermined value can be a range, for example, where the general population is divided equally (or unequally) into groups, or into quadrants, the lowest quadrant being individuals with the lowest levels of systemic marker, the highest quadrant being individuals with the highest levels of systemic marker.

The predetermined value can be derived by determining the level of TSHR mRNA in the general population. Alternatively, the predetermined value can be derived by determining the level of TSHR mRNA in a select population. Accordingly, the predetermined values selected may take into account the category in which an individual falls. Appropriate ranges and categories can be selected with no more than routine experimentation by those of ordinary skill in the art.

Predetermined values of TSHR mRNA, such as for example, mean levels, median levels, or “cut-off” levels, are established by assaying a large sample of individuals in the general population or the select population and using a statistical model such as the predictive value method for selecting a positively criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for each systemic marker that is assayed.

The preoperative (i.e., prior to thyroidectomy) levels of TSHR mRNA detected in the subject's bodily sample may be compared to a single predetermined value or to a range of predetermined values to categorize the thyroid neoplasia in the subject, i.e., determine whether thyroid neoplasia is benign or malignant, and the extent of the disease. Preoperative levels of TSHR mRNA in the test subject's bodily sample that are higher than a predetermined value or range of predetermined values can be indicative of the subject having malignant lesions. Preoperative levels of TSHR mRNA in the test subject's bodily sample lower than a predetermined value or range of predetermined values can be indicative of the subject having benign thyroid neoplasia, such as those associated with follicular adenoma.

The postoperative levels (i.e., following thyroidectomy) of TSHR mRNA detected in the subject's bodily sample may also be compared to a single predetermined value or to a range of predetermined values to the recurrence of thyroid cancer. Postoperative levels of TSHR mRNA in the test subject's bodily sample that are higher than a predetermined value or range of predetermined values can be indicative of recurrent/residual thyroid cancer. Postoperative levels of TSHR mRNA in the test subject's bodily sample that are lower than a predetermined value or range of predetermined values can be indicative of an absence of thyroid cancer and spare the subject unnecessary surgical intervention.

The present invention is further directed to a kit for identifying and detecting TSHR mRNA in a bodily sample by means of a nucleic acid based assay. The kit includes at least one pair of oligonucleotide primers. The pair of oligonucleotide primers can include nucleic acid sequences that are specific to TSHR mRNA and that can be used to amplify a target nucleotide sequence, which is defined by contiguous nucleotides from, for example, exons 6-9 of TSHR mRNA.

In one example of the present invention, the kit comprises a pair of oligonucleotide primers. The oligonucleotide primers include at least 10 contiguous nucleotides that are capable of selectively amplifying exons 6-9 of TSHR mRNA. By way of example, the oligonucleotide primers can have nucleic acid sequences comprising SEQ ID NOs: 1 and 2. Optionally, the kit may also contain one or all of the reagents necessary to begin the PCR amplification reaction and fluorescent detection of the oligonucleotide probes.

In accordance with another aspect of the invention, the method of detecting thyroid cancer in a subject can comprise detecting circulating thyroid cells in a bodily sample of a subject by obtaining an appropriate nucleic acid sample from the bodily sample of the subject and determining whether the nucleic acid sample includes thyroglobulin (Tg) mRNA.

The presence Tg mRNA can be determined by detecting a target nucleotide sequence of the Tg mRNA. By way of example, a portion of the nucleic acid sequence that comprises Tg mRNA, which can be used as a target nucleotide sequence in accordance with the present invention, comprises exons 1 to 5 of Tg mRNA.

In an aspect of the invention, the target nucleotide sequence can be detected by amplifying a target nucleotide sequence of the nucleic acid sequence that comprises mRNA corresponding to the reverse transcript of DNA encoding Tg and detecting the amplified target sequence. The target nucleotide sequence of the present invention can be amplified by selectively hybridizing oligonucleotide primers that facilitate transcription and replication of at least a portion of the Tg mRNA (or a reverse transcript of the Tg mRNA).

Oligonucleotide primers capable of specifically (or selectively) hybridizing to the Tg mRNA in accordance with the present invention can comprises at least about 10 nucleotides. By way of example, the oligonucleotide primers can comprise about 10 to about 40 nucleotides, and more particularly about 15 to about 35 nucleotides. The oligonucleotide primers can be of sufficient length and complementary with a portion of the nucleotide sequence of the Tg mRNA to form a duplex with sufficient stability for the purpose intended. For example, the oligonucleotide primers should contain a nucleic acid sequence of sufficient length and complementarity to the targeted Tg mRNA to allow the polymerizing agent to continue replication from the primers, which are in stable duplex form with the target sequence, under polymerizing conditions.

An example of a pair of nucleic acid sequences that can be used for the pair of nucleotide primers include SEQ ID NOs: 3 and 4. SEQ ID NO: 3 is a forward primer that comprises the following nucleotide sequence: 5′AGGGAAACGGCCTTTCTGAA 3′ (SEQ ID NO: 3). SEQ ID NO: 4 is a reverse primer that comprises the following nucleotide sequence: reverse 3′, CTTTAGC-AGC-AGAAGAGGTG 5′ (SEQ ID NO: 4). SEQ ID NOs: 3 and 4 when used as oligonucleotide primers are capable of amplifying a 407 base pair segment of exons 1-5 of Tg mRNA.

It will be appreciated by one skilled in the art that other oligonucleotide primers of the present invention can include nucleic acid sequences complementary to SEQ ID NOs: 3-4, nucleic acid sequence substantially similar to SEQ ID NOs: 3-4, nucleic acid sequences substantially similar to a nucleic acid sequence complementary to SEQ ID NOs: 3-4, a fragment of SEQ ID NOs: 3-4 that specifically hybridize to the Tg mRNA, a fragment of a nucleic acid sequence complementary to SEQ ID NOs: 3-4 that specifically hybridize to Tg mRNA, a fragment of a nucleic acid sequence substantially similar to SEQ ID NOs: 3-4 that specifically hybridizes to the Tg mRNA, and a fragment of a nucleic acid sequence substantially similar to nucleic acid sequences complementary to SEQ ID NOs: 3-4 that specifically hybridizes to the Tg mRNA. It will also be appreciated that the oligonucleotide primers can include other nucleic acid sequences as long as these nucleic acid sequences specifically hybridize to Tg mRNA, and specifically amplify exons 1-5 of the Tg mRNA.

The nucleic acids used to form the Tg mRNA oligonucleotide primers in accordance with the present invention can be derived from Tg mRNA. The Tg mRNA oligonucleotide primers in accordance with the present invention can be made by methods well known in the art, such as chemical synthesis. The primers may be synthesized manually or by machine. They may also be synthesized by recombinant methods using products incorporating viral and bacterial promoters.

The oligonucleotide primers can be used in an amplification assay that detects at least a portion of the Tg mRNA. In an aspect of the invention the oligonucleotide primers can be used in a polymerase chain reaction (PCR) assay. It will appreciated by one skilled the art that the Tg mRNA oligonucleotide primers in accordance with the invention as well as other nucleic acids complementary to Tg mRNA nucleic acids can also be used in other assays, such as a RAPD assay or other amplification assay.

The amplified target nucleotide sequence, if present, can then detected using known quantitative and/or qualitative detection techniques, such as visualization of restriction enzyme digestion patterns determined by gel electrophoresis, sequencing of the amplified target nucleotide sequence, detection of the amplified nucleotide sequence with an oligonucleotide hybridization probe.

The preoperative (i.e., prior to thyroidectomy) levels of Tg mRNA detected in the subject's bodily sample may be compared to a single predetermined value or to a range of predetermined values to categorize the thyroid neoplasia in the subject, i.e., determine whether thyroid neoplasia is benign or malignant, and the extent of the disease. Preoperative levels of Tg mRNA in the test subject's bodily sample that are higher than a predetermined value or range of predetermined values can be indicative of the subject having malignant lesions. Preoperative levels of Tg mRNA in the test subject's bodily sample lower than a predetermined value or range of predetermined values can be indicative of the subject having benign thyroid neoplasia, such as those associated with follicular adenoma.

The postoperative levels (i.e., following thyroidectomy) of Tg mRNA detected in the subject's bodily sample may also be compared to a single predetermined value or to a range of predetermined values to the recurrence of thyroid cancer. Postoperative levels of Tg mRNA in the test subject's bodily sample that are higher than a predetermined value or range of predetermined values can be indicative of recurrent/residual thyroid cancer. Postoperative levels of Tg mRNA in the test subject's bodily sample that are lower than a predetermined value or range of predetermined values can be indicative of an absence of thyroid cancer and spare the subject unnecessary surgical intervention.

The present invention is further directed to a kit for identifying and detecting Tg mRNA in a bodily sample by means of a nucleic acid based assay. The kit includes at least one pair of oligonucleotide primers. The pair of oligonucleotide primers can include nucleic acid sequences that are specific to Tg mRNA and that can be used to amplify a target nucleotide sequence, which is defined by contiguous nucleotides from, for example, exons 1-5 of Tg mRNA.

In one example of the present invention, the kit comprises a pair of oligonucleotide primers. The oligonucleotide primers include at least 10 contiguous nucleotides that are capable of selectively amplifying exons 1-5 of Tg mRNA. By way of example, the oligonucleotide primers can have nucleic acid sequences comprising SEQ ID NOs: 3 and 4. Optionally, the kit may also contain one or all of the reagents necessary to begin the PCR amplification reaction and fluorescent detection of the oligonucleotide probes.

Those skilled in the art will also understand and appreciate variations in the method in accordance with the present invention. For example, it is to be appreciated that the methods of the present invention can be used in conjunction with other detection methods known in the art, such as FNA. Moreover, it will be appreciated that the present method can be used as a means to monitor the efficacy of therapeutic agents used to treat thyroid carcinoma. These therapeutics can be administered in conjunction prior to after a thyroidectomy

The present invention is illustrated by the following examples. The examples are set forth to aid in an understanding of the invention, but are not intended to, and should not be construed to, limit in any way the invention as set fort in the claims, which follow hereafter.

### EXAMPLES

### Example 1

Detection of TSH-Receptor mRNA and Thyroglobulin mRNA Transcripts in Peripheral Blood of Patients with Thyroid Disease: Sensitive and Specific markers for Thyroid

Thyroglobulin production by both normal and neoplastic thyroid tissues depends on the presence of functional TSH receptors (TSHR), and is influenced by TSH levels. We investigated the sensitivity and specificity of TSHR-mRNA and Tg-mRNA detection by RT-PCR in the peripheral blood from normal subjects and from patients with thyroid cancer and benign thyroid diseases.

Methods

Subjects

A total of 153 patients including 51 normal subjects without a history of thyroid disease (females:males=1.7; age range 25-60 years), 27 with benign thyroid disease (female:male=3.5; age range 18-77 years) and 75 patients with differentiated thyroid cancer (DTC) (female:male=3.2; age range 20-80 years), were evaluated. Among the 27 patients with benign thyroid disease, three patients had thyroiditis, 18 had solitary thyroid nodules or multinodular goiters, three had primary hypothyroidism on replacement T₄ therapy and three had Graves' disease.

Among the DTC patients, 67 (89%) had a near-total thyroidectomy and in whom 65 (83%), had had radioactive iodine (RAI) ablation at least one year prior to mRNA testing. The remaining eight patients, all with newly diagnosed papillary thyroid cancer, were tested prior to thyroidectomy. All DTC patients were evaluated during visits as outpatients in the Department of Endocrinology, Diabetes and Metabolism at the Cleveland Clinic Foundation. A chart review was conducted to obtain each patient's history, operative/pathology reports and laboratory and radiological examinations.

The pathology, disease status, treatment status and Tg antibody status of 75 patients with thyroid cancer are listed in Table 1.

TABLE 1
Characteristics of thyroid cancer patients

| | | | Number of Patients tested (number with | | | | | |
|---|---|---|---|---|---|---|---|---|
| | | | disease) by pathologic diagnosis | | | | | |
| Treatment | | Tg Ab | Papillary | | Follicular | | Hürthle | |
| status | N | positive | Ca | | Ca | | cell Ca | |
| Treated: | | | | | | | | |
| T4 Therapy | 49 | 12 | 40 | (12) | 6 | (3) | 3 | (3) |
| T4 Withdrawal | 7 | 2 | 6 | (1) | 1 | (0) | 0 | (0) |
| After rhTSH | 11 | 0 | 11 | (0) | 0 | (0) | 0 | (0) |
| Newly | | | | | | | | |
| Diagnosed: | | | | | | | | |
| Pre Surgery | 8 | 0 | 8 | (8) | 0 | | | |
| Total | 75 | 14 | 65 | (21) | 7 | (3) | 3 | (3) |

Forty-nine patients were studied while on T₄ suppression, seven after T₄ withdrawal and 11 after the administration of recombinant human TSH (rhTSH) (Thyrogen, Genzyme Transgenics Corp., Cambridge, Mass.). Among 49 patients evaluated during T₄ therapy, 41 (86%) had a diagnostic radioactive-iodine scan within 12 months from the date of testing and four patients within 24-48 months prior to the testing; all were monitored with serum thyroglobulin determinations and were considered free of disease if the scan was negative and/or they had undetectable Tg levels. Four had no available scans; two of them had recurrent disease (one with pulmonary and one with node metastasis), one had negative ultrasonography and lymph node biopsies; and the other had undetectable serial serum Tg levels and no clinical evidence of disease. None of the patients received RAI therapy after their last whole body-scan (WBS) except in three with known metastatic disease as confirmed by other imaging procedures and pathological examination.

Blood samples for TSHR and Tg mRNAs were collected at various intervals from the initial date of surgery. Concurrent serum levels of TSH (Roche Diagnostics NJ) and Tg (Quest Diagnostics CA) were measured by immuno-chemilumino-metric assay (ICMA) and the sensitivity was defined as 1.0 μg/L. Tg antibodies were also measured in most patients by enzyme immunoassay (EIA; Tosoh Medics Inc. CA). A Tg value of ≧1.0 μg/L in patients tested during T₄ therapy and a value of ≧2.0 μg/L in patients tested after rh-TSH administration or after T₄ withdrawal was considered to be a significant indicator for follow-up WBS (24). All patients on T₄ suppression had TSH values <1.0 mU/L (61% had TSH<0.1) except in five with TSH between 1.0-2.8 mU/L. All patients studied after T₄ withdrawal had TSH values greater than 30 mU/L except one (TSH of 19 mU/L).

Our RAI scanning procedure included measurement of neck uptake 24 hours after administration of a tracer dose (100 μCi) of ¹³¹I and a diagnostic WBS obtained at 48 hours after administering a 5 mCi dose of ¹³¹I. Patients tested after rh-TSH (0.9 mg intramuscularly for two days) had a 5 mCi dose of ¹³¹I administered, 24 hr after the second dose of rhTSH and a WBS obtained 48 hrs thereafter. Blood samples for both TSH/Tg mRNA as well as Tg measurement were drawn at the same time prior to scan. Scans were reviewed by the Nuclear Medicine physicians and were considered positive if these showed visible uptake in the thyroid bed and/or discrete focal uptake was present at sites that normally did not pick up ¹³¹I. Patients were classified as having evidence of disease if they had a positive WBS or disease diagnosed by pathology or found by other non-radioiodine imaging modalities.

RT-PCR

Approximately 3-5 mL of whole blood (collected in EDTA-treated tubes) was mixed with equal volume of PBS pH 7.4, layered with 8 ml Ficoll (Pharmacia) and centrifuged at 400×g for 20 minutes at 4° C. The mononuclear cell layer was collected, washed and pelleted. RNA was isolated using Trizol Reagent (Invitrogen CA) following the manufacturer's instructions. Optical density ratio of A_{260/280} was used to assess the quality and quantity of isolated RNA. One-two μg total RNA was reverse transcribed to cDNA using Superscript Pre-amplification System (Invitrogen CA) following the instruction manual.

PCR was performed using the selected primer pairs. The primer sequences were: TSHR: forward 5′GCTTTTCAGGGACTATGCAA-TGAA 3′ (SEQ ID NO: 1); and reverse 3′AGAGTTTGGTCAC-AGTGACGGGAA 5′ (212 bp) (SEQ ID NO: 2); Tg: forward 5′AGGGAAACGGCCTTTCTGAA 3′ (SEQ ID NO: 3); reverse 3′, CTTTAGC-AGC-AGAAGAGGTG 5 (407 bp)′ (SEQ ID NO: 4); Glyceraldehyde-3-phosphate dehydrogenase (GAPDH), a control gene ubiquitously expressed, was also analyzed to confirm the success of RNA extraction and RT and PCR reactions using primers as previously reported. PCR was carried out for 38 cycles (94° C. for 1 minute (first cycle for 2 minutes), 62° C. for 1 minute, 72° C. for 1 minute (10 minutes for the last cycle). RT-PCR products were resolved on 2% gel electrophoresis and visualized by ethidium bromide staining. The estimated sensitivity for these assays was tested by serial dilution of thyroid cancer tissue RNA with RNA obtained from normal peripheral blood mononuclear cells and was found to be ˜10 cancer cells/mL of blood.

Statistical Analysis

Data were analyzed for diagnostic sensitivity and specificity of TSHR mRNA and Tg mRNA, serum Tg and WBS for detection of recurrent/residual thyroid cancer. The comparisons of sensitivity and specificity between the markers were performed using Fisher Exact Test. P<0.05 was considered as significant.

Results

TSHR-mRNA in Normal Controls and Benign Thyroid Disease:

Results are summarized in Table 2. All fifty-one of the normal controls were negative for both TSHR and Tg-mRNA. Of the patients with benign thyroid disease, 24 of 27 (89%) were negative for both TSHR and Tg-mRNA, including 15 patients with thyroid nodules. Of the three positive patients, two had massive obstructive goiters (100 and 300 gm, 10×8×3 cm and 11×11×5 cm) and the third had a follicular adenoma. All three patients had their diagnosis confirmed by surgical pathology.

TABLE 2
TSHR mRNA and Tg mRNA positivity in healthy euthyroid
subjects, benign thyroid disease and in thyroid cancer
patients without evidence of disease

| | | Number Positive (%) | | | | |
|---|---|---|---|---|---|---|
| Subjects | N | TSH-R mRNA | | Tg-mRNA | | Serum Tg* |
| Normal | 51 | 0 | | 0 | | NA† |
| Benign Thyroid | 27 | 3 | (11)** | 3 | (11)** | NA† |
| Disease | | | | | | |
| TC with no | 48 | 1 | (2) | 4 | (8.5) | 2 (4) |
| Evidence of Disease | | | | | | |

| | | | | | |
|---|---|---|---|---|---|
| Total Positives/Total | 4/125 | (97%) | 7/125 | (94%) | — |
| (% Specificity) | | | | | |

*Using a cut off of ≧1 μg/L on T₄ therapy; ≧2 μg/L after T₄ withdrawal or rh TSH.
**2 patients with massive obstructive goiters and 1 with a follicular adenoma
†NA = not applicable

TSHR mRNA in Patients with Thyroid Cancer.

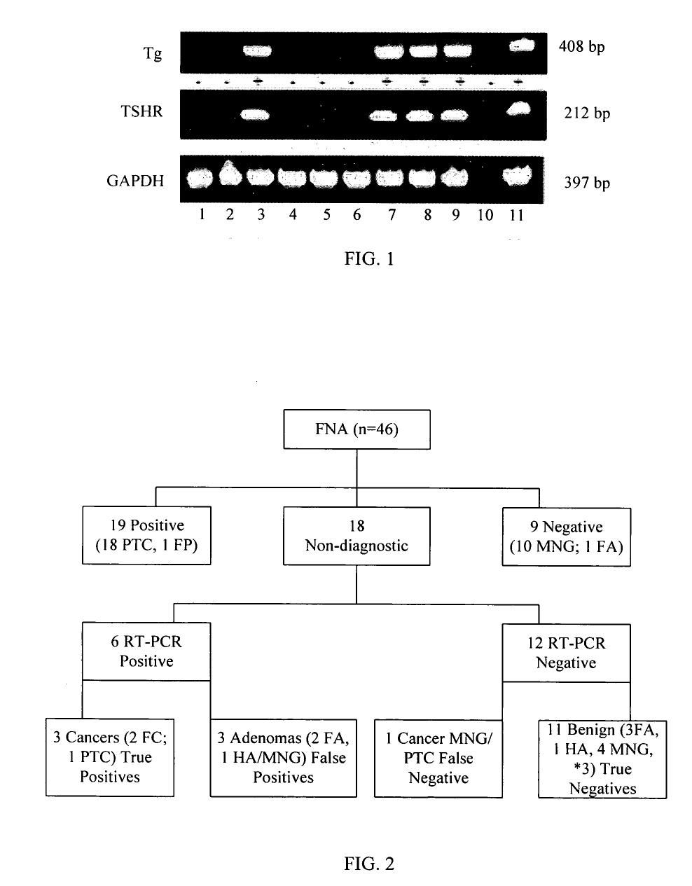

FIG. 1 shows the representative RT-PCR products for TSHR (212 bp), Tg (407 bp) and GAPDH (397 bp) as obtained in nine thyroid cancer patients, one normal subject and in a positive thyroid cancer control. Table 3 summarizes the results obtained in the 67 previously treated DTC patients and in the eight patients with DTC tested prior to surgical resection.

TABLE 3
TSHR mRNA, Tg mRNA and serum Tg levels
in patients with thyroid cancer

| | | No. positive (%) | | | |
|---|---|---|---|---|---|
| Patients | (N) | TSHR mRNA | Tg-mRNA | Tg | I¹³¹ Scan |
| Treated: | | | | | |
| On T₄ | | | | | |
| Suppression | | | | | |
| Distant | 8 | 8(100) | 8(100) | 8(100) | 6(75) |
| Metastasis | | | | | |
| Local | 10 | 10(100) | 10(100) | 9(90) | 6(75)** |
| Metastasis | | | | | |
| No Disease | 31 | 0(0) | 2(6) | 0 | 0(0)*** |
| After T₄ | | | | | |
| Withdrawal | | | | | |
| Local | 1 | 1(100) | 1(100) | 1(100) | 1(100) |
| Metastasis | | | | | |
| No Disease | 6 | 1(17) | 1(17) | 1(17) | 0(0) |
| After | | | | | |
| rh-TSH | | | | | |
| No Disease | 11 | 0(0) | 1(9) | 1(9) | 0(0) |
| Untreated: | | | | | |
| Pre-Sugery | 8 | 6(75) | 6(75) | NA† | NA† |

*Using a cut off of ≧1 μg/L on T₄ therapy; ≧2 μg/L after T₄ withdrawal or rh TSH.
**N = 8;
***N = 29;
†NA = not applicable

Forty-nine patients with TC were tested while on T₄ therapy. Eight of these patients had distant metastases, 10 had local recurrences or cervical lymph node disease and the remaining 31 were disease free. Table 3 lists the % positives as obtained with TSHR-mRNA, Tg-mRNA, serum Tg levels and WBS. Both TSHR and Tg-mRNA were positive in all patients with distant metastases or local disease. Also serum Tg detected all but one patient with recurrent disease while on T₄ suppression. Three of seven follicular and all three Hurthle cell cancer patients with evidence of disease were positive for both TSHR and Tg-mRNA.

Of the 31 patients with no evidence of disease, TSH-R mRNA was detected in none and Tg-mRNA was detected in two (6%). These two patients had undetectable serum Tg values and negative WBS and remained disease-free at 1-year follow up.

Seven patients were tested after T₄ withdrawal. One had evidence of local recurrence on WBS and was positive for both TSH-R and Tg-mRNA. Of the six patients with no evidence of disease on WBS, one was positive for both TSH-R and Tg-mRNA but had an undetectable serum Tg value after T₄ withdrawal. Another had a post-withdrawal serum Tg value of 55.5 ng/ml (negative antibodies; negative for TSH-R and Tg mRNA) but one year later was found to have an undetectable Tg value, negative WBS and negative thyroid ultrasound on T₄ therapy. The cause of this isolated high Tg level is not clear.

Eleven patients were tested after rh-TSH. None had evidence of disease. One was positive for Tg-mRNA but negative for TSH-R-mRNA and WBS This patient had a serum Tg value of 2.3 ng/ml after rh-TSH. One year later, the serum Tg after rh-TSH was undetectable despite no treatment having been given. Subsequent WBS and three consecutive Tg levels after thyroid withdrawal have been negative so far and patient has been considered as having no evidence of disease.

Tg-mRNA in Patients with Anti-Tg Antibodies:

Fourteen (21%) of the patients with DTC had anti-Tg antibodies. Eleven of the 14 patients had no evidence of disease; all had an undetectable serum Tg level (measured values are spuriously low in the presence of Tg antibodies) and all were negative for TSH-R-mRNA. All three patients with local disease were positive for both TSHR and Tg mRNAs including one which was negative for serum Tg.

Diagnostic Performance of TSH-mRNA

Table 4 summarizes the diagnostic performance characteristics of TSHR-mRNA, and compares these with Tg-mRNA, serum Tg levels as well as with WBS to detect recurrent/metastatic disease.

TABLE 4
Diagnostic performances of TSH-R mRNA, Tg mRNA, serum Tg level and
¹³¹I uptake/WBS in previously treated thyroid cancer patients
A. TSHR mRNA positivity: comparison with Tg mRNA, serum Tg and WBS

| | Number Positive/Total (%) | | | |
|---|---|---|---|---|
| Patients | TSH-R mRNA | Tg mRNA | Serum Tg | ¹³¹I uptake/WBS |
| Evidence of Disease | 19/19(100) | 19/19(100) | 18/19(95) | 13/17(76) |
| No Evidence of Disease | 1/48(2) | 4/48(8) | 2/48(4) | 0/46(0) |

| | | | |
|---|---|---|---|
| Concordance with TSHR mRNA | 64/67(95) | 64/67(95) | 59/63(94) |

B: Performance Characteristics

Diagnostic Performance

| | TSH-R mRNA | Tg mRNA | Serum Tg | ¹³¹I uptake/WBS |
|---|---|---|---|---|
| Sensitivity | 100% | 100% | 95% | 83% |
| Specificity | 98% | 92% | 96% | 100% |
| PPV | 95% | 83% | 90% | 100% |
| NPV | 100% | 100% | 96% | 94% |
| Efficiency | 98% | 94% | 94% | 95% |

There were no statistically significant differences among these markers and both TSHR-mRNA and Tg-mRNA had equal sensitivity for detection of residual/recurrent disease (P=0.209, Fisher exact test). Two patients with NED (negative scan and subsequent undetectable Tg levels) had elevated serum Tg levels, one post T₄ withdrawal and another post rh-TSH, but were negative for both TSHR-mRNA and Tg-mRNA. A WBS was not done in four patients and was negative in three patients with evidence of disease (two with lung metastases and one with node metastases (sensitivity=82%). The concordances between TSHR-mRNA and Tg-mRNA and between TSHR-mRNA and serum Tg (in Tg antibody negative patients) were both 95%.

Discussion

The major finding in the present report is that the presence of TSHR-mRNA signals in blood is specific for patients with thyroid cancer being undetectable in healthy subjects and in the vast majority of patients with benign thyroid diseases. Furthermore, we demonstrated the high specificity of Tg-mRNA for thyroid cancer when carefully selected primers are used in the assay. Thyroid carcinomas are known to contain functional TSH receptors. To date, this target has not been exploited for detection of circulating cancer cells, perhaps due to previous reports showing the presence of TSHR-mRNA transcripts in peripheral blood mononuclear cells and other extrathyroidal tissues. The finding of these transcripts in extrathyroidal tissues can be explained by TSHR splice variants. Thus, selection of primers specific to thyroid cells is of paramount importance in the assay.

There was a high concordance between the TSH-R and Tg mRNA in our series. These Tg-mRNA findings contrast with a number of previous studies using both qualitative and quantitative RT-PCR, which detected Tg-mRNA signals in most normal subjects. As discussed previously, these differences are most likely due to the primer pair selection rather than differences in assay sensitivities or other variables in methodologies. Indeed, we previously detected signals in controls, as well, using some primer pairs described in the literature. It is possible that, with certain primer pairs, amplification of pseudogenes can give rise to false positive results. A more likely explanation for this discordance is the limitation of PCR-based techniques in their capability of detecting alternative splice variants amplified by the selected primers.

Whereas other investigators have reported a much higher incidence (20-33%) of Tg mRNA positivity in patients with benign thyroid disease, we obtained false positive results in only three out of 27 (11%) patients. One of these false positives was a patient who was found to have a follicular adenoma on surgical pathology. The differentiation between follicular adenoma and follicular carcinoma is at present only possible following surgical resection and formal histological examination. To date, there are no known markers that can distinguish follicular adenomas form cancer with certainty since, like follicular cancer, a significant number of follicular adenomas harbor Ras mutations and show galectin-3 immuno-staining. Therefore, the finding of circulating thyroid cells in a patient with follicular adenoma is not unparalleled and favors the notion that some follicular adenomas may represent a pre-malignant stage of follicular carcinomas. The other two false positives were in patients with massive goiters indicating that such patients may have circulating thyroid cells. Alternatively, an occult DTC may have been overlooked in the pathology examination. Regardless, since most other benign nodules are negative and six out of eight DTC patients tested preoperatively were positive, this test may have a potential use in the differential diagnosis of cancer from benign thyroid nodules preoperatively.

Our results indicate that TSHR-mRNA by RT-PCR is a highly sensitive and specific marker in monitoring patients for recurrent or metastatic thyroid cancer. Our data also show the high sensitivity and reliability of serum Tg levels to detect most recurrent disease while patient is on T₄ suppressive therapy. Therefore, RT-PCR assay may not prove to be a cost-effective alternative for serum Tg levels as a first line of testing. Its value lies in patients in whom Tg measurements are not reliable due to the presence of interfering Tg antibodies, heterophile antibodies or other factors. In our series, TSHR mRNA or Tg mRNA was detected in all patients with local or distant metastases, tested while on T₄ therapy or after thyroid hormone withdrawal. Serum Tg levels were elevated (≧2.0 ng/mL) in all but one (Tg antibody positive) of these patients in whom the need of WBS would have been indicated by positive mRNA testing. Furthermore, the finding of a negative TSHR or Tg-mRNA signal might have obviated the need for a WBS in two Tg positive patients and in all Tg antibody positive patients with no evidence of disease.

Among the 48 DTC patients who had no evidence of disease, TSHR mRNA was positive (2%) less often than Tg mRNA (8%). None of these positive patients had the disease at a one-year review as evidenced by serum Tg levels and/or WBS. Nonetheless, these patients deserve to be monitored carefully since they may harbor microscopic disease that is being detected earlier by the Tg-mRNA assay.

Unlike previous reports, which find Tg mRNA only in patients with papillary carcinoma but not in other histologic types, we found no differences in either Tg or TSH mRNA results based on tumor histology. Among our patients, three of seven follicular carcinoma patients and all three Hürthle cell carcinoma patients, with evidence of disease were positive for both TSHR and Tg-mRNA.

In summary, the presence of either TSHR-mRNA or Tg mRNA in peripheral blood is specific for the presence of residual/recurrent DTC disease and is as sensitive as serum Tg in monitoring Tg antibody-negative patients. However, in Tg antibody-positive patients with unreliable serum Tg values, TSHR-mRNA or Tg mRNA surveillance may prove to be more cost-effective by obviating the need for a WBS in mRNA negative patients. Furthermore, the high specificity of mRNA testing combined with our preliminary findings of its ability to detect thyroid cancer preoperatively suggests a potential role in screening patients with thyroid nodules.

### Example 2

Thyrotropin Receptor/Thyroglobulin Messenger Ribonucleic Acid in Peripheral Blood and Fine-Needle Aspiration Cytology: Diagnostic Synergy for Detecting Thyroid Cancer

Patients and Methods

Patients

A total of 72 consecutive patients carrying the diagnosis of thyroid disease that is benign, malignant, or of as yet undetermined malignant potential, who were referred to our endocrine surgery clinic and who had signed informed consent form, were enrolled in this Institutional Review Board-approved study. All 72 patients had a preoperative sample drawn either during preoperative laboratory testing or on the day of surgery. Ultrasound-guided FNA biopsies were performed in 46 of 72 patients as a routine diagnostic work-up, and when not performed at our institution, the cytology slides were obtained and reviewed by one of our experienced pathologists specializing in thyroid cytology. Criteria for FNA sample adequacy included sufficient number of cells, abundant colloid, and the presence of at least six groups of benign follicular cells composed of at least 10 cells each. The results were compared with final postoperative pathological diagnosis.

RT-PCR

Methods we have previously described in detail in Example 1 were used. Briefly, 5 ml of venous blood was collected, and mononuclear cells were separated using Ficoll Hypaque gradient within 24 h after collection. RNA was extracted from the mononuclear cells immediately after the Ficoll separation using TRIzol reagent (Life Technologies, Carlsbad, Calif.), and 1 μg was reverse transcribed with Superscript II preamplification system (Life Technologies). PCR was performed using carefully selected primers based on specificity (no illegitimate transcription), as documented in our previous publications. PCR was carried out for a total of 38 cycles [94 C for 1 min (first cycle for 2 min), 62 C for 1 min, and 72 C for 1 min (10 min for the last cycle)]. PCR products generated with RNA from a thyroid cancer tissue and from a peripheral blood sample (DTC patient) were sequenced with ABI-PRISM 310 genetic analyzer (Applied Biosystems, Foster City, Calif.) using their BigDye Terminator v3.1 sequencing kit. The sequence of the transcript was identical to TSHR mRNA sequence, confirming the presence of authentic receptor mRNA. Glyceraldehyde-3-phosphate dehydrogenase was used as a control for successful RNA extraction and transcription and PCRs. RT-PCR products were resolved on 2% gel electrophoresis and visualized by ethidium bromide staining. Gel images were captured in “live mode” automated setting (integration/exposure time, 0.2 sec) with the use of Gel-doc 1000 (Bio-Rad, Hercules, Calif.) system and software.

Data Analysis

TSHR mRNA and FNA results were compared with final pathological diagnoses. The number of patients correctly classified (diagnostic accuracy) by TSHR/Tg mRNA or by FNA singly or in combination was calculated and compared using x² test.

Results

A total of 72 patients were enrolled in the study (61 females and 11 males; age range, 18-88 yr; mean, 50 yr). Forty-six of these patients had FNA biopsy performed before surgery. Postoperative pathological diagnosis was used to categorize the patients into benign and malignant thyroid disease groups (Table 5). Sixty-one patients (25 DTC and 36 benign) were enrolled at initial diagnosis (with no prior thyroid surgery). Eleven patients had recurrent or residual malignant disease at the time of enrollment, and 10 of these had prior radioactive iodine ablation.

TABLE 5
Patients Categorized According to Final Pathological Diagnosis

| Thyroid Cancer | | Benign Thyroid Disease | |
|---|---|---|---|
| Cancer Histology/Surgery | Positive/Total | Pathological Diagnosis | Negative/Total |
| Papillary | 26/33 (79%) | Thyroid Adenoma | 5/8 (62.5%) |
| Thyroid only | 9/13 | Follicular | 4/6 |
| Thyroid + LN | 7/10 | Hurthle Cell | 1/2 |
| LN/Neck Dissection | 10/10 | MNG/nodules | 17/20 (85%) |
| Follicular Carcinoma | 3/3 (100%) | Graves/Thyroiditis | 7/8 (87.5%) |
| Thyroid + LN | 2/2 | Negative/total (% specificity) | 29/36 (80%) |
| LN/Neck Dissection | 1/1 | | |
| Positive/Total (% Sensitivity) | 29/36 (80%) | | |

LN, Lymph nodes.

There was 100% concordance between RT-PCR results for Tg and for TSH-R mRNA. FIG. 1 shows the representative RT-PCR products for TSHR (212 bp) and Tg (408 bp) in seven patients. A total of 36 patients were positive by RT-PCR, including 29 of 36 (sensitivity 80%) cancer patients and 7 of 36 (specificity 80%) benign disease patients. Among 25 patients with initial diagnosis, 19 were positive by RT-PCR (sensitivity 72%) (Table 5). All false negatives had pathological diagnosis of papillary thyroid carcinoma (PTC) and included three patients with lymph node metastasis. False positives included two patients with follicular adenoma (FA), one patient with Hurthle cell adenoma, one patient with hyperplastic oxyphilic nodule, and three patients with very large multinodular goiters (MNG).

Of the 46 patients with FNA, 22 had surgically confirmed DTC. Eighteen of these 22 (82%) had a definitive FNA result for PTC, and four patients, including two follicular carcinoma (FC) and two PTC in MNG, had sufficient specimen but inconclusive results. Of the 24 patients with surgically confirmed benign disease undergoing FNA, nine were definitively benign (including three patients with large MNG), and 14 were sufficient but inconclusive (Table 6). Overall 18 of 46 (39%) patients had adequate specimen but inconclusive/indeterminate FNA biopsy results. The diagnostic sensitivity of FNA was 82%, but overall diagnostic accuracy (efficiency) was only 61%. Results are summarized in Table 6.

TABLE 6
Diagnostic Performance of FNA and TSHR mRNA
in Patients With No Previous Thyroid Surgery

| | Thyroid Cancer | | | Benign Thyroid Disease | | | |
|---|---|---|---|---|---|---|---|
| | | | Sensitivity/ | | | Specificity/ | Diagnostic |
| Test | N | Positive | PPV (%) | N | Negative | NPV (%) | accuracy (%) |
| mRNA | 22 | 16 | 73/76 | 24 | 19 | 79/76 | 76 |
| FNA | 22 | 18 (4)^{α} | 82/95^{b} | 24 | 9 (14)^{α} | —/—^{c} | 59 |
| mRNA and FNA | 22 | 21 | 95/84 | 24 | 20 | 83/95 | 89 |

PPV, Positive predictive value;
NPV, Negative predictive value.
^{α}Adequate sample but nondiagnostic FNA.
^{C}alculated using four patients with indeterminate FNAs as negatives.
^{c}Not calculated due to large number of nondiagnostic FNAs.

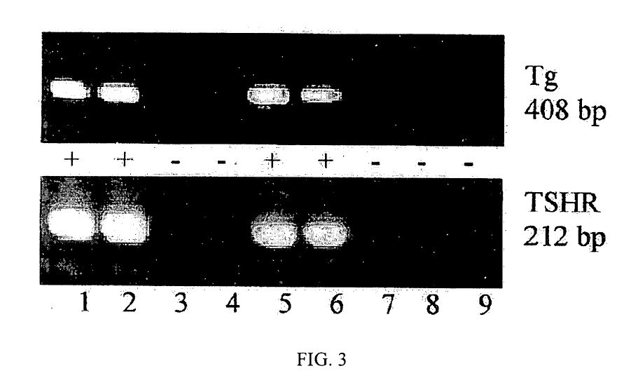

When FNA was indeterminate on a sample sufficient for cytological diagnosis (18 patients), RT-PCR correctly classified three of four cancer patients and 11 of 14 benign disease patients (sensitivity 75%; specificity 78%). Only one patient with PTC foci in MNG was negative (false negative). The actual FNA results and RT-PCR results on these 18 patients with equivocal FNA are summarized in FIG. 3. The combined diagnostic sensitivity (95%) and diagnostic efficiency (89%) for RT-PCR and FNA was significantly higher than FNA alone (P=0.001).

Discussion

In this study, 39% of FNA biopsies were called indeterminate. According to previous reports, the vast majority (78%) of these were indeed found to have benign histology. These indeterminate thyroid nodules seem to be one of the most frustrating and challenging areas for endocrinologists and endocrine surgeons. Most studies are focused on FNA material to identify molecular markers or a combination of markers as means of improving the accuracy of diagnosis made by FNA. A reliable and satisfying method that is able to differentiate preoperative malignant potential in patients presenting with thyroid nodules has not yet been proposed.

Our results suggest that circulating preoperative TSHR/Tg mRNA acts as an adjunctive test to enhance the diagnostic accuracy of FNA and classified 78% (14 of 18) of nodules with indeterminate FNA accurately. There were only three false-positive patients and one false-negative patient with TSHR/Tg mRNA among the FNA indeterminate group. It is recognized that FNA cytology has a high sensitivity for PTC. Also, in this series, FNA correctly identified all except two patients with equivocal results. In comparison, RT-PCR had relatively low sensitivity for PTC and was negative in seven patients, including three patients with lymph node metastasis. Factors responsible for these false-negative results remain unclear at present and may include technical errors and sampling problems, or they may relate to inefficient reverse transcription or nonspecific inhibitors of the PCR. Among these factors, the technical error is less likely because repeat analysis using a second PCR produced the same results. It is possible that the efficiency of reverse transcription or PCR may be the limiting factor in this assay, and these factors are currently being investigated in our laboratory.

On the other hand, as expected, FNA cannot differentiate between FC and FA. In this study, we had seven patients with follicular neoplasms, and FNA was nondiagnostic for all except one. Hence, follicular neoplasms are often grouped together as indeterminate or follicular patterned thyroid lesions and require a substantial number of FNAs and unnecessary diagnostic thyroid lobectomies. To date, there are no known markers that can distinguish FAs from cancer with certainty because, like FC, a significant number of FAs harbor Ras mutations and show galectin-3 immunostaining. It is suggested that some FAs may represent a premalignant stage of FCs. We demonstrated the high sensitivity of circulating TSHR mRNA in detecting recurrent/residual disease in patients with all DTCs regardless of histological types, including follicular and Hurthle cell cancers. In this study, this test detected both patients with FCs as positive and three of five FA as negatives, thus correctly classifying five of seven follicular neoplasms. Although the number of follicular neoplasms is small in this series, our results indicate a high potential of TSHR mRNA to differentiate FC from FA and await confirmation in future studies with a larger number of patients. Besides two FAs, other false positives were one MNG with hyperplastic nodules, predominantly Hurthle cell type, and three patients with extremely large MNG, suggesting that such patients may have circulating thyroid cells. This may be due to the presence of high hyperplastic activity in thyroid nodule, or it is possible that an occult DTC may have been present and was overlooked in the pathology examination.

In conclusion, our results demonstrate that circulating TSHR/Tg mRNA has lower sensitivity to detect PTC than FNA at initial diagnosis. However, TSHR/Tg mRNA shows promise for detecting FC, which is often missed by FNA. Its value resides in identifying benign thyroid disease among patients with equivocal FNA. Overall, it may serve as a valuable adjunct to FNA for identifying thyroid cancer from benign disease.

### Example 3

Clinical Evaluation of Enhanced Qualitative and Quantitative RT-PCR Assays for TSHR mRNA in Peripheral Blood for Preoperative Diagnosis of Malignancy in Patients with Thyroid Nodular Disease

In this study, we continue the investigation of the clinical utility of this promising molecular marker by using more sensitive qualitative and quantitative RT PCR assays and to detect and quantify the levels of TSHR, pre and post-operatively on each newly diagnosed thyroid cancer patient referred for surgical resection and in consecutive patients who undergo FNA for thyroid nodules.

Our hypothesis is that, more sensitive quantitative determinations of TSH receptor mRNA in the peripheral blood may further enhance the detection of cancer cells. Which in turn when combined with the thyroid FNA biopsies will allow for more accurate classification of thyroid nodules into benign and malignant lesions. Furthermore, the quantitative levels may indicate the extent of disease preoperatively and may serve as a marker for residual/metastatic disease post-operatively. This should result in improved selection of patients for thyroid surgery and, thus spare many patients unnecessary surgical intervention.

Materials and Methods

Blood Collection/Isolation of Mononuclear Cells and of Circulating Epithelial Cells

Six ml venous blood is collected in EDTA-treated tubes, placed on ice, and processed as soon as possible. Whole blood is mixed with equal volume of PBS pH 7.4, layered with 8 ml Ficoll (Pharmacia) in a 15 ml polystyrene tube. The samples are centrifuged at 400×g for 20 min. at 4° C. The mononuclear cell layer is collected, washed with PBS pH 7.4, and pelleted.

RNA Extraction

RNA is isolated using Trizol Reagent (Life Technologies) following manufacturer's instructions. Briefly, Trizol Reagent is added to the mononuclear cell pellet, and incubated for 5 min. at room temperature. Chloroform extraction is then carried out. RNA in the aqueous phase is precipitated with isopropanol, washed with 75% ethanol, dried and resuspended in DEPC-treated water. Optical density ratio of A_{260/280} is used to assess the quality and amount of isolated RNA

Enhanced Qualitative One Step Reverse-Transcription (cDNA Synthesis) and PCR

The Superscript III one step RT-PCR system with Platinum® Taq DNA polymerase (Invitogen Inc) will be used according to manufacturer's instructions. Our preliminary data shows significant enhancement of sensitivity with use of this more efficient RT system compared to previous use of superscript II two-step procedure. In brief, 25 μl reaction mixture containing 1 μg total RNA, 10 μM primers, Superscript III RT/platinum Taq high fidelity enzyme mix (1 μL) and 25 μL of autoclaved distilled water will be placed in thermocycler. The 1st cycle will be programmed for cDNA synthesis and pre-denaturation and consists of 55° C. for 30 minutes followed by 94° C. for 2 min. PCR will be performed for 40 cycles of denature at 94° C. for 15 seconds, anneal at 60° for 30 seconds and extend at 68° C. for 1 minute and reaction will be terminated at 70° C. for 5 minutes.

Real-Time PCR Assay

One step “Quantitech Syber Green RT-PCR” procedure and reagents from Qiagen Inc. CA will be used. Briefly, 25 μL of SYBR Green RT-PCR master mix, 2 μM (1 μL) of each primer and 500 ng (0.5 μL) of RNA template will be added to PCR tubes. The thermocycler will be programmed for cDNA synthesis at 50° C. for 30 minutes and initial inactivation step at 95° C. for 15 minutes then followed by 1^{st} PCR cycle of denature at 94° C. for 15 seconds, anneal at 60° for 30 seconds and extend at 72° C. for 30 seconds for total of 40 cycles. Melting curve analysis of the RT-PCR product will be performed to verify the specificity and to identify the RT-PCR product. Total RNA preparation from thyroid cancer tissue will be used as reference preparation to generate a standard curve and to achieve the relative quantification. Also simultaneously GAPDH a house-keeping gene will be measured as endogenous control and will be used to correct for sample to sample variations in RT-PCR efficiency and for RNA loading amounts.

Patient Population

1. Patients presented with thyroid nodules: 100 consecutive patients presented with thyroid nodules on whom, FNA is performed will be tested prior to FNA.
2. Newly Diagnosed Thyroid Cancer: 50 patients with new diagnosis of thyroid cancer referred to our surgery department will be tested before and day after surgery.
3. Pediatric thyroid cancer: 10-20 patients, younger than 18 years of age presented with thyroid nodules requiring FNA biopsy will be tested before and after surgery.
   Statistical Analysis (Compiled with the Help of Ed Mascha, Dept. of Biostatistics) Sample Size Calculations

Based on our previous experience with less sensitive qualitative RT-PCR we have obtained about 75% sensitivity and about 90% specificity with this assay. With 50 patients referred for surgery (group 1) and 100 patients who present with thyroid nodule to endocrinology the expected number of thyroid cancer cases is about 40-70 patients and number of cases not having thyroid cancer will be about 80-110 patients. We will be able to obtain an accurate estimate of sensitivity that has a 95% confidence interval (CI) about 0.66-0.90 wide and of specificity that has CI of 0.15-0.20 wide or better.

Planned Data Analysis

For all analyses the gold standard for deciding whether patient has thyroid cancer or benign thyroid disease will be based on pathologic diagnosis by FNA biopsy or final tissue pathology. Group 1 patients will be those with newly diagnosed thyroid cancer (positive or equivocal FNA). Of these we estimate that about 60-100% will test positive with the RT-PCR assays. From this group we will obtain estimates and 95% confidence intervals of sensitivity. Group 2 will be patients who come to clinic for evaluation of thyroid nodules and undergo for FNA biopsies. Among this group we estimate about 5-30% may be positive for cancer. Here we will obtain estimates and 95% confidence intervals of specificity. Data from both groups will be used to calculate both positive and negative predictive values (PPV, NPV) and efficiency of TSHR mRNA for thyroid cancer detection.

### Example 4

Quantitative Reverse-Transcription-Polymerase Chain Reaction (RT-PCR) Measurement of Thyroid-Stimulating-Hormone Receptor Messenger RNA (TSHR-mRNA) in the Peripheral Blood of Patients with Thyroid Cancer

Assay methods based on RT-PCR of thyroid specific-mRNA may improve monitoring for thyroid cancer recurrence. We have previously shown that qualitative RT-PCR for TSHR-mRNA can be sensitive and specific for recurrent thyroid cancer. Here, we developed and validated a quantitative RT-PCR assay using an in-cycle fluorescent detection system (SYBR Green 1; Rotorgene 3000™). We studied 59 patients; 15 healthy subjects, 19 patients with benign thyroid disease, 14 patients with newly diagnosed thyroid cancer (13 papillary, 1 follicular) and 11 patients with recurrent thyroid cancer (all papillary). Blood samples were taken pre-operatively, and all diagnoses of thyroid disease were confirmed by surgical pathology. mRNA was extracted from venous blood and quantitated using a spectrophotometer. One step RT-PCR was performed according to manufacturers recommendations for the Quantitect SYBR Green kit (Qiagen) and PCR was carried out for 40 cycles. Gel electrophoresis confirmed the identity of amplified products. Total RNA extracted from a surgical specimen of papillary thyroid carcinoma was serially diluted to create a standard curve to calibrate the assay. The intraassay coefficient of variation (CV) (n=8) for the threshold cycle was 1.4% and the mean interassay CV (n=12) was 2.7%. The samples were normalized for the amount of RNA loaded into each reaction tube (1 g) and quantified using the thyroid cancer RNA standard curve. Results are reported as pg TSHR-mRNA/g thyroid cancer mRNA (Table 7). The results suggests a significant difference between the TSHR-mRNA levels in patients without thyroid cancer (normal patients/benign thyroid disease), compared to patients with thyroid cancer (newly diagnosed/recurrent).

TABLE 7

| | | Significance (p) |
|---|---|---|
| | Median | compared to |
| Group | TSHR-mRNA(range) | normal subjects |
| Normal | 0.07(0-0.26) | — |
| Benign thyroid disease | 0.1(0-31.76) | 0.57 |
| Newly diagnosed thyroid | 32.54(5.85-69.75) | <0.0001 |
| cancer | | |
| Recurrent thyroid cancer | 33.58(4.81-103.45) | <0.0001 |

Differences between the patient groups were tested with the Wilcoxon Rank Sum test

### Example 5

Measurement of TSH Receptor mRNA by Qualitative RT-PCR in Peripheral Blood: Role of Pre and Post Surgery Levels in Monitoring Patients with Thyroid Cancer

Qualitative RT-PCR for TSHR-mRNA is a sensitive and specific marker for recurrent thyroid cancer. To assess its value in monitoring patients after surgery, we measured TSHR-mRNA levels by a quantitative RT-PCR. A total of 71 subjects (26 normal; 19 benign thyroid disease; 15 newly diagnosed and 11 recurrent thyroid cancer) were studied. TSHR-mRNA levels were measured pre and on the first post-operative day, with 17 patients having further follow-up levels (mean follow-up 19.1±10 months). The status of residual/metastatic disease was assessed at 9-18 months after surgery by stimulated Tg levels (ng/mL) and/or whole body I 131 scan (WBS). The upper limit of normal subjects (0.78 ng/μg total RNA; median 0.12) defined the cutoff level for a positive mRNA test. The medians (range) for pre/post-op levels for benign disease were 0.22 (0-27.3)/0.23 (0-0.57) and for newly diagnosed and recurrent cancer patients were 29.8 (0.07-69.7)/0.03 (0.01-9.5) and 33.58 (4.8-52.9)/0.15(0.03-1365) respectively. Among 15 patients with newly diagnosed thyroid cancer, 14 became negative for TSHR-mRNA post-operatively. All remained disease free on follow-up except one, who had a positive stimulated Tg level (13.6 ng/mL) but was WBS negative and had no clinically relevant disease on further imaging. One patient (Tg antibody positive) remained positive post-op and was actually missed by WBS and stimulated Tg and had clinically relevant disease diagnosed by pathology. Among 11 patients with recurrent thyroid cancer, 7 became negative by our assay post-op; 4 of these remained disease free on follow-up and the remaining 3 had increased stimulated Tg levels (=<9.3 ng/mL) but negative WBS, and had no further treatment. Four of 11 patients remained positive by assay post-op; all had metastatic disease and elevated Tg=>46 ng/mL and positive WBS. Overall concordance with stimulated Tg was 81% and with WBS was 96%.

Our results suggest that TSHR-mRNA has a short life in circulation and post-op levels in patients with thyroid cancer are predictive of residual/metastatic disease.

Having described the invention, the following is claimed:

1. A method of detecting thyroid cancer in a subject, the method comprising:
obtaining a nucleic acid sample from a bodily sample of the subject; and
determining whether the nucleic acid sample contains thyroid stimulating hormone receptor (TSHR) mRNA.

2. The method of claim 1, the THSR mRNA being determined by amplifying a segment of TSHR mRNA in the nucleic acid sample; and
detecting the presence of the amplified segment of TSHR mRNA in the bodily fluid sample.

3. The method of claim 2, the amplification being performed with a pair of primers that are complementary to the TSHR mRNA.

4. The method of claim 3, the segment of TSHR mRNA amplified by the primers spanning at least a portion of exons 6-9 of TSHR mRNA.

5. The method of claim 4, at least one primer comprising SEQ ID NO: 1.

6. The method of claim 4, at least one primer comprising SEQ ID NO: 2.

7. The method of claim 1, the bodily sample comprising blood.

8. A preoperative assay for determining whether thyroid neoplasia in a subject is benign or malignant, comprising:
obtaining a nucleic acid sample from peripheral blood of the subject; and
determining whether the nucleic acid sample contains thyroid stimulating hormone receptor (TSHR) mRNA.

9. The method of claim 8, the THSR mRNA being determined by amplifying a segment TSHR mRNA in the nucleic acid sample; and
detecting the presence of the amplified segment of TSHR mRNA in the bodily fluid sample.

10. The method of claim 9, the amplification being performed with a pair of primers that are complementary to the TSHR mRNA.

11. The method of claim 10, the segment of TSHR mRNA amplified by the primers spanning at least a portion of exons 6-9 of TSHR mRNA.

12. The method of claim 11, at least one primer comprising SEQ ID NO: 1.

13. The method of claim 11, at least one primer comprising SEQ ID NO: 2.

14. A method of detecting thyroid cancer in a subject, the method comprising:
obtaining a nucleic acid sample from a bodily sample of the subject;
amplifying a segment of TSHR mRNA in the nucleic acid sample; and
detecting the presence of the amplified segment of TSHR mRNA in the bodily fluid sample.

15. The method of claim 14, the amplification being performed with a pair of primers that are complementary to the TSHR mRNA.

16. The method of claim 15, the segment of TSHR mRNA amplified by the primers spanning at least a portion of exons 6-9 of TSHR mRNA.

17. The method of claim 14, the primers comprising at least one of SEQ ID NO: or SEQ ID NO: 2.

18. The method of claim 14, the TSHR mRNA being detected using gel electrophoresis.

19. The method of claim 14, the detection of TSHR mRNA being indicative of malignant or metastatic thyroid cancer.

20. A preoperative assay for determining whether thyroid neoplasia in a subject is benign or malignant, comprising:
obtaining a nucleic acid sample from a bodily sample of the subject;
amplifying a segment of Tg mRNA in the nucleic acid sample using a pair of primers, at least one of the primers comprising SEQ ID NO: 3 or SEQ ID NO: 4; and
detecting the presence of the amplified segment of Tg mRNA in the bodily fluid sample.

21. The method of claim 20, the Tg mRNA being detected using gel electrophoresis.

22. The method of claim 20, the detection of Tg mRNA being indicative of malignant or metastatic thyroid cancer.

23. The method of claim 20, the bodily sample comprising peripheral blood.
